(19) United States
(12) Patent Application Publication
(10) Pub. No.: US 2012/0245614 A1
(43) Pub. Date: Sep. 27, 2012

(54) Branch and Truncal Vessel Occluder

(76) Inventor: William Joseph Drasler, Minnetonka, MN (US)

(21) Appl. No.: 13/423,108

(22) Filed: Mar. 16, 2012

Related U.S. Application Data
(60) Provisional application No. 61/465,544, filed on Mar. 21, 2011.
(60) Provisional application No. 61/518,267, filed on May 3, 2011.

(51) Int. Cl. A61M 29/00

(52) U.S. Cl. 606/191

(57) ABSTRACT

A low profile occlusion device for percutaneous or direct transcutaneous placement into a vein or other tubular vessel of the body. Two wires form one or more loops having a thin cover disposed between them. The loop forms a beveled angle within the blood vessel and the cover prevents blood flow through the vessel. An additional loop can be present to prevent device embolization or device migration. The occlusion device is delivered to a vein such as an incompetent perforator vein or other incompetent vein through a small flexible sheath. Ultrasound guidance is used to position the occlusion device which can be repositioned within the vessel if necessary.

### CROSS REFERENCE TO RELATED APPLICATIONS

This nonprovisional patent application makes reference to and includes information found in the provisional patent applications No. 61/465,544 entitled “Branch and Truncal Vessel Occluder” filed 21 Mar. 2011 and 61/518,267 entitled “Branch and Truncal Vessel Direct Occluder” filed 3 May 2011, both by William J. Drasler.

### FIELD OF THE INVENTION

This invention relates to an implanted device that is placed percutaneously or transcutaneously into a lumen of a tubular member of the body such as a vein or artery or a side branch of a vein or artery to occlude the lumen of the tubular member.

### BACKGROUND OF THE INVENTION

Superficial veins either below or above the knee can develop incompetent valves with an inability to direct blood from the superficial venous system into the deep system and result in reflux of blood from the deep system into the superficial system. Such reflux can occur at the junction of a superficial vein such as the saphenous vein with the deep veins such as the femoral vein or the popliteal vein. Also reflux can occur between the superficial venous system and the deep system through incompetent valves associated with perforator veins. This reflux of blood can result in varicose veins and superficial venous insufficiency.

One way to treat this problem is by ablating the superficial veins. Several devices and methods have been used to ablate the saphenous vein including endovascular laser therapy (EVLT) and endovascular radiofrequency therapy (EVRFT). Such devices are expensive and are modestly effective in maintaining long term occlusion of mid-sized saphenous veins of the upper leg, but are not as effective in providing occlusive therapy for perforator vessels or smaller truncal veins of the lower leg. Another technique for occluding such veins is by use of sclerotherapy. With sclerotherapy a liquid sclerosant or a foam sclerosant is injected into the vein to cause trauma to the endothelial lining of the vessel resulting in occlusion of the vein. Such systems are difficult to control and can result in the sclerosant flowing into the deep venous system potentially causing trauma to this important deep system which is necessary for venous return of blood from the leg to the heart.

Another way of treating incompetent perforator veins is via subfascial endoscopic perforator vein surgery (SEPS) wherein access is made via two large endoscopes to the site of the perforator vein and an external clip is placed on the vein to occlude it. This procedure is expensive and requires that the patient be placed on general anesthesia; it is very infrequently performed.

Additionally, occlusion coils can be placed intravascularly into the perforator vein to occlude them via a percutaneous procedure. This procedure is required to be performed under fluoroscopy to ensure that the coils are not embolized into the deep venous system. The use of fluoroscopy adds cost to the procedure, along with concomitant risks associated with the use of contrast medium and exposure to x-ray radiation.

Ligation of perforator veins via direct surgery is not very practical due to the difficulty to access these vessels and the potential problems associated with a surgical procedure. Also, many patients have compromised healing in the lower leg including venous ulcers, thereby causing these patients to be unsuitable for surgical perforator ligation. Surgical ligation of truncal veins would be considered more traumatic than necessary, especially with access to current modalities such as EVLT and RVT for treatment of truncal varicosities.

What is needed is an occlusion device that can be delivered percutaneously under Duplex guidance to block or occlude perforator veins. The device is required to have a very low profile to fit within the small diameter perforator veins and to be very flexible to make the sharp turn, approximately 90 degrees from the truncal superficial vein into the perforator vein or from the deep vein into the perforator vein. The occlusion device must provide the assurance that under Duplex guidance, it will not be delivered to the deep venous system and cannot embolize into the deep system. Under the circumstance that the truncal superficial vein is itself competent, the device should only occlude the perforator vein and leave the truncal vein to remain patent. If the truncal vein is incompetent, the device should be able to contribute to the occlusion of the truncal vein.

### SUMMARY

The present invention is a percutaneously delivered device for occluding the lumen of a vein or artery or other vessel lumen of the body. The primary embodiment is well suited to occlude an incompetent perforator vein that extends from a superficial truncal vein such as the greater saphenous vein (GSV) or smaller saphenous vein (SSV) or a deep vein (30) such as the femoral or popliteal vein. The invention can also be used to occlude any side branch of a vessel that extends from a truncal vessel or to occlude the truncal superficial vessel itself. Perforator veins located in the upper and lower leg are intended to deliver blood from the superficial veins to the deep veins for return of blood back to the heart.

The occlusion device is comprised of a Nitinol or elastomeric metal frame that is formed into a looped shape or a figure-eight shape that it is not planar; this portion of the device is located in the branch vein or perforator vein. Each loop of the figure-eight is beveled with respect to the axis of the vessel during implant to cause the edges of the frame to come into contact with the perforator vein wall around its perimeter and adjust to the diameter of the vein. The adjustment occurs by the changing the bevel angle of the loop with respect to the axis of the vein. Each loop of the branch portion is covered by a material that is generally occlusive to blood flow and can have small pores that will allow the material to become ingrown with cells over time. Such materials include expanded polytetrafluoroethylene (ePTFE), porous polyurethanes, and others including biological tissue coverings such as pericardial tissues, cellular films, collagen films, fibrin films and others; the pore size can range from nonporous to pores of dimensions in the size range of a micrometer to over 30 micrometers.

The frame of the present invention has a very low profile in its non-deployed configuration consisting of two frame members or wires that are aligned adjacent to each other. The low profile associated with these two wires along with their very large flexibility allows the present occlusion device to enter into small diameter veins and achieve a turn from one vein into another.

One embodiment also has a second truncal portion attached to the branch portion that is intended to be delivered to the truncal vein such as the saphenous vein to which the perforator vein is joined. The truncal portion is also formed of a Nitinol frame. In a preferred embodiment the truncal frame is contiguous with the branch frame such as a continuous metal wire or a contiguous metal shape. The truncal frame has a dimension that extends approximately perpendicular to the axial direction of the perforator vein and has a truncal portion length that is greater than the deployed branch portion diameter thereby prohibiting the truncal portion from entering the perforator vein. This prevents the occlusion device from embolizing into the deep venous system or migrating downstream into the truncal vein.

The truncal portion can have a similar figure-eight structure to the branch portion and can have an occlusive material attached to the frame. The presence of an occlusive material can help to cause an incompetent truncal vein to occlude and prevent the recurrence of reflux in the superficial vein. Alternately, the truncal frame can have an open structure to allow blood to continue to flow freely in a fully competent truncal superficial vein or in a deep vein.

The occlusion device is delivered in a low profile delivery sheath that holds the occlusion device in a small diameter configuration. The delivery sheath is able to travel over a guidewire that is first passed, for example, into the superficial vein, through the incompetent perforator vein, and into the deep venous system. The delivery sheath containing the occlusion device is passed over the guidewire and into the perforator vein using Duplex ultrasound to help guide the passage. With the branch portion contained within the delivery catheter extending into the perforator vein, the delivery sheath is withdrawn as a pusher tube is held in position to release the branch portion of the occlusion device into the perforator vein. Upon verification of positioning via an ultrasound marker on the delivery catheter and on the occlusion device the truncal portion of the device is released into the truncal vein. The branch vessel is then occluded under Duplex ultrasound guidance without fear of embolization of the occlusion device into the deep venous system. The occlusion device can be alternately introduced percutaneously into a deep vein of the leg and directed into the incompetent perforator vein rather than introducing it from the superficial vein.

In an alternate embodiment the truncal portion can be attached to the branch portion via a connector that allows rotation between the two portions while maintaining the two portions in a generally perpendicular configuration with respect to each other. The truncal portion can extend across the diameter of the truncal vessel to gain support from the opposite wall of the truncal vein to provide a force onto the branch portion to ensure that the branch portion of the occlusion device is not able to dislodge into the superficial vein.

In yet another embodiment, the truncal portion is a linear member or a more linear looped member that extends substantially in the axial direction of the truncal vein. It is understood that for any of the embodiments of the present invention the branch portion of the occlusion device can consist of two loops of a figure-eight as previously stated or it can have only one loop, less than one full loop, or more than two loops and still fall within the understanding of the present invention. Also, the branch portion of the occlusion device is required to make contact along its perimeter with the perimeter of the vein with which it is in contact, contain an occlusive material attached to the frame, be able to automatically adjust in diameter to changing diameters for the vein, and be attached to a second or truncal portion that has a dimension that is larger in length than the diameter of the vein in which the branch portion resides, to fit within the teachings of this patent application.

It is understood that any embodiment of the present invention can be used to directly occlude a truncal vessel such as a saphenous vein alone rather than a branch vessel. The longer length of the truncal portion in a direction perpendicular to the axis of the branch portion will restrict migration of the occlusion device in a generally straight or tortuous truncal vessel.

In still another embodiment, the occlusion device of the present invention can be comprised of only a single portion similar to the branch portion and not have a second or truncal portion with a generally perpendicular dimension as described earlier. The occlusion device can consist, for example, entirely of a figure-eight shape that is not planar and having an occlusion material attached to the frame. This occlusion device can be placed into a truncal vein or branch vessel at any location that it is desired for an occlusion to occur. For example, it can be of benefit to occlude the GSV near the saphenofemoral junction to ensure that thrombus does not extend into the deep venous system potentially causing deep venous thrombosis (DVT) to occur. Such an occlusion device could be placed at any location that an incompetent venous valve exists in the truncal superficial venous system of the leg or other incompetent veins of the body. It is understood that the figure-eight shape could assume another shape that makes contact of the perimeter of a frame with the vessel wall and has an occluding material and is able to adjust in diameter to the changing diameters of the vein from sitting to standing and from rest to full activity.

It is understood that the devices of the present invention can be delivered to a perforator vein through an access site made transcutaneously or through the skin directly adjacent to the perforating vein. A needle can be advanced directly through the skin and into the lumen of the perforator vein. A guidewire can then be advanced through the needle and into the perforator. A catheter or delivery sheath containing the occluder device can then be advanced over this guidewire directly through the skin and into the perforator vein for delivery into the perforator vein or branch vessel.

### BRIEF DESCRIPTION OF THE DRAWINGS

FIG. 1A is a plan view of the occlusion device having two branch loops and two truncal loops in a deployed configuration extending from a truncal vessel into a branch vessel.

FIG. 1B is a cross sectional view of the branch vessel with the branch portion of the occlusion device residing within.

FIG. 2 is a plan view of the occlusion device located within the delivery sheath.

FIG. 3 is a plan view of the occlusion device having two branch loops deployed within the branch vessel and two truncal loops located within the delivery sheath.

FIG. 4 is a plan view of the occlusion device deployed with two truncal loops located on the proximal and distal sides of the branch vessel.

FIG. 5 is a plan view of an occlusion device having two truncal loop that contact the opposite wall of the truncal vessel.

FIG. 6 is an embodiment of the occlusion device with a low profile truncal loop.

FIG. 7A is an embodiment of the occlusion device having a single branch loop and a single truncal loop.

FIG. 7B is a cross sectional view of truncal portion of the device shown in FIG. 7A.

FIG. 8 is a plan view of an embodiment of the occlusion device having a very short axial length for the branch portion and having three loops for the truncal portion.

FIG. 9A is a plan view of an embodiment of the occlusion device that is placed entirely within either the branch vessel or a truncal vessel.

FIG. 9B is a plan view of an embodiment of the occlusion device having three loops located within either a branch vessel or a truncal vessel.

FIG. 9C is a plan view showing the location of an ultrasound marker on the delivery sheath and an ultrasound marker on the occlusion device.

### DETAILED DESCRIPTION OF THE INVENTION

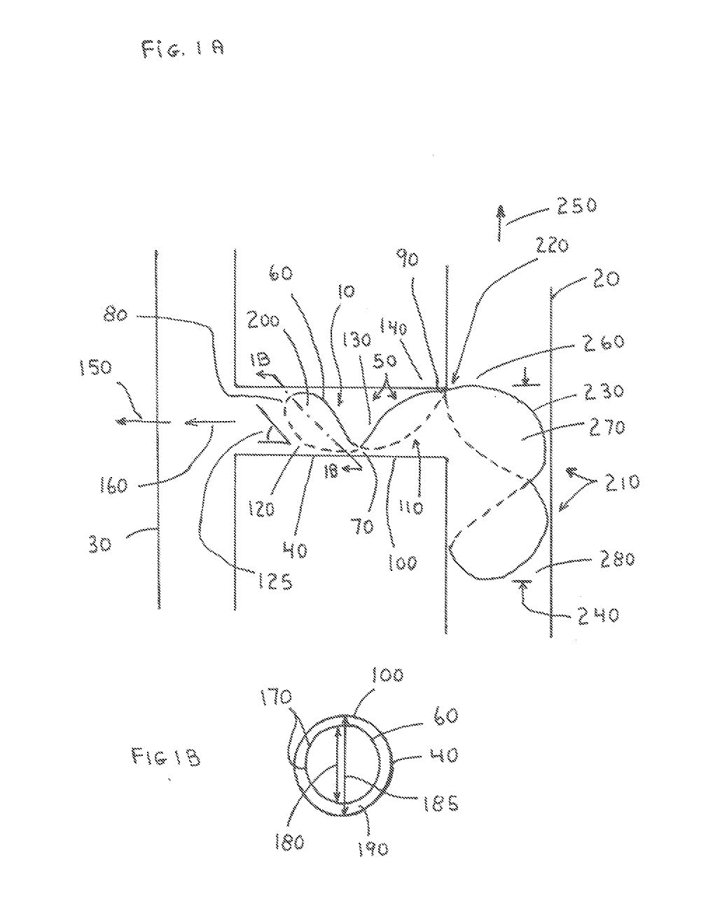

The present invention is an occlusion device (10) (FIG. 1A) that is passed percutaneously or transcutaneously into a truncal vessel (20) or deep vein (30) and delivered to a perforator vein or other branch vessel (40) that extends from the truncal vessel (20) or is delivered directly into a truncal vessel (20) or branch vessel (40) to occlude such vessel directly. Access to the truncal vessel (20) can be from a distal site such as a GSV or a SSV near the ankle or access can be from a proximal site into the GSV or SSV near the inguinal fold or near the knee. The device can also be delivered percutaneously to an artery or other tubular member of the body to create an occlusion.

FIGS. 1A and 1B show a first embodiment of the present invention as it applies to occlusion of a perforator vein or other branching vessel (40) off of a truncal vein such as a saphenous vein or other truncal vessel (20). The occlusion device (10) is comprised of a branch portion (50) that extends into the perforator vein (40). The branch portion (50) is comprised of a figure-eight shaped branch frame (60) that is bent such that the figure-eight is not planar; instead, it is curved such that the branch crossover (70) of the figure-eight frame resides along the vessel wall (100). The distal end (80) and proximal end (90) of the branch frame (60) is in contact with the vessel wall (100) approximately 180 degrees opposite to the branch crossover (70). The branch frame (60) forms two branch loops, a proximal branch loop (110) and a distal branch loop (120) that are thus held at a beveled angle (125) from the distal end (130) to the proximal end (140) of each loop with respect to the perforator vein axis. Each loop of the branch frame (60) has approximately the same beveled angle (125). The beveled angle (125) for each loop of the branch frame (60) can differ from each other without deviating from the teaching of the present invention. The perimeter (170) of each loop of the figure-eight is held into contact with the vessel wall (100) even if the perforator vein diameter changes due to changes in hydrostatic pressure. As the diameter of the vein changes, the bevel angle of the branch loops will adjust to continue to make contact of its frame perimeter (170) with the vessel wall (100). The beveled angle (125) can range from 20-70 degrees off of the branched vessel axis or the branched portion axis which forms the axis of the branch frame (60) after deployment and is perpendicular to the effective diameter (180) of the branch frame (60) and branch vessel (40). The preferred range for the beveled angle (125) is from 30-60 degrees off of the branched vessel or branched portion axis.

The end view down the perforator vein in FIG. 1B shows the intimate contact of the branch frame (60) of the distal branch loop (120) making contact with the vessel wall (100) along the entire branch frame perimeter (170). The branch portion effective diameter (180) is the diameter of the expanded frame when projected in a direction aligned with the axis (150) of the blood vessel; the branch portion effective diameter (180) is substantially equal to the branch vessel diameter (185) after the frame has made contact with the vein and stretched it an amount ranging from 2-100% of the normal vein diameter as observed under average venous pressure. The proximal branch loop (110) and distal branch loop (120) both have an effective diameter (180) that is equal to the branch portion effective diameter (180). Each loop of the branch portion (50) makes contact with the branch vessel (40) along a perimeter (170) to form a seal (190) generated by intimate contact that will occlude the branch vessel (40). The diameter (185) of a branch vessel (40) that is a perforator vein can range from 2-7 mm and typically would range from approximately 3-5 mm.

The branch frame (60) is formed from a single branch frame element or branch frame member (60) that is formed from an elastic metal such as Nitinol or elgiloy, that is able to retain elastic energy such that the branch frame (60) can push outwards at the branch frame distal end (80) and branch frame proximal end (90) of the occlusion device (10) against the vessel wall (100) to form a seal (190) with the vein and prevent leakage of blood around the occlusion device (10). The frame element or frame member (60) can be formed from a Nitinol wire, for example, and the wire diameter can range from 0.003-0.014 inches. The branch frame (60) can also be formed of stainless steel, or other materials commonly used to make stents for blood vessels including biodegradable polymers and metals. The frame member or frame element (60) in a preferred embodiment is formed from a single elastic fiber such as a metal fiber that forms one or more loops.

An occlusive branch cover (200) is attached to the branch frame (60) of the branch portion (50) of the occlusion device (10). The branch cover (200) extends across the branch portion effective diameter (180) to block flow of fluid through the blood vessel This branch cover (200) can be formed from expanded polytetrafluoroethylene (ePTFE), electrostatically spun polyurethane, other porous polyurethane or silicone fibrous films, or other implantable flexible and thin materials including biodegradable films used in medical devices, tissue membranes, collagen membranes, fibrin membranes, and others. The branch cover (200) should be generally impervious to blood flow but should allow cellular penetration to occur such that complete healing can occur on each side of the branch cover (200). The branch cover (200) can be attached to the branch frame (60) for example by thermally melting a plastic cover to itself as it surrounds the branch frame (60). Alternately, solvents and adhesives can be used to bond a plastic branch cover (200) directly to the branch frame (60) or to itself around the branch frame material. The branch cover (200) should be attached to the branch frame (60) in a folded configuration or in a manner that allows the branch cover (200) to extend in area as the branch frame (60) extends outward to make contact with the branch vessel (40).

The curved figure-eight shape for the branch portion (50) will continue to form a seal (190) with the vessel wall (100) without the tendency for migration if it is exposed to pressure or flow from the deep venous system or from the superficial system. The beveled angle (125) of the distal branch loop (120) and proximal branch loop (110) provide a triangular stability against movement of the occlusion device (10). The beveled angle (125) of each loop will adjust to changes in vessel diameter by altering its beveled angle (125) to maintain contact of the branch frame (60) and the perimeter of each loop with the vessel wall (100).

Attached to the branch portion (50) of the occlusion device (10) of one embodiment is a truncal portion (210) forming a branch/truncal junction (220). The truncal frame (230) is either contiguous with the branch frame (60) or it can be attached via a connector, via welding, adhesives or via a swiveling connector. A connector can be a small length of wire or metal or other material that is used to join to both the branch and truncal portions (210) or is contiguous with both the branch and truncal portions (210). The truncal frame (230) of the truncal portion (210) can be formed of similar material to the branch frame (60). In some embodiments, the truncal frame (230) can be formed from plastic materials used in medical device implants or biodegradable materials such as polyglycolic acid, polylactic acid, magnesium, and others.

The truncal frame (230) has a truncal length (240) in its deployed configuration in the direction of the truncal portion axis (250) which is approximately perpendicular or at a significant angle (more than 45 degrees) to the branch portion axis (160) that is greater than the branch portion effective diameter (180) in its deployed configuration and greater than the branch vessel diameter (185). As shown in FIG. 1A, the truncal frame distal end (260) and branch frame proximal end (90) are joined or are contiguous end to end but the angle of the branch portion axis (160) is approximately perpendicular (range 45-90 degrees) to the truncal portion axis (250). This approximate perpendicular angle helps to prevent the truncal portion (210) from entering into the perforator vein and prevents the branch portion (50) from backing out into the truncal vessel (20). The result is that the occlusion device (10) is unable to embolize into the deep vein (30) or further into the superficial system or truncal vessel (20). The truncal length (240) extends from the truncal frame proximal end (280) to the truncal frame distal end (260).

The truncal frame (230) can have an occlusive truncal cover (270) attached to it in a manner similar to the branch cover (200). For this embodiment the truncal portion (210) will assist in creating an occlusion in the truncal vessel (20) which is desirable in situations where the truncal vessel (20) has incompetent valves and blood is refluxing in the superficial veins of the leg towards the foot. Materials for construction for the truncal cover (270) are similar to that described for the branch cover (200).

Alternately, in a separate embodiment, the truncal frame (230) does not have an occlusive truncal cover (270) on it. Instead, the blood is free to flow in an antegrade direction which is the case for fully competent valves in the truncal superficial veins. The truncal frame (230) can be formed with a nonplanar figure-eight configuration or other configuration that places the frame entirely into contact with the truncal vessel wall (100) such that blood flow is not impeded. The materials for the truncal frame (230) are similar to those described for the branch frame (60).

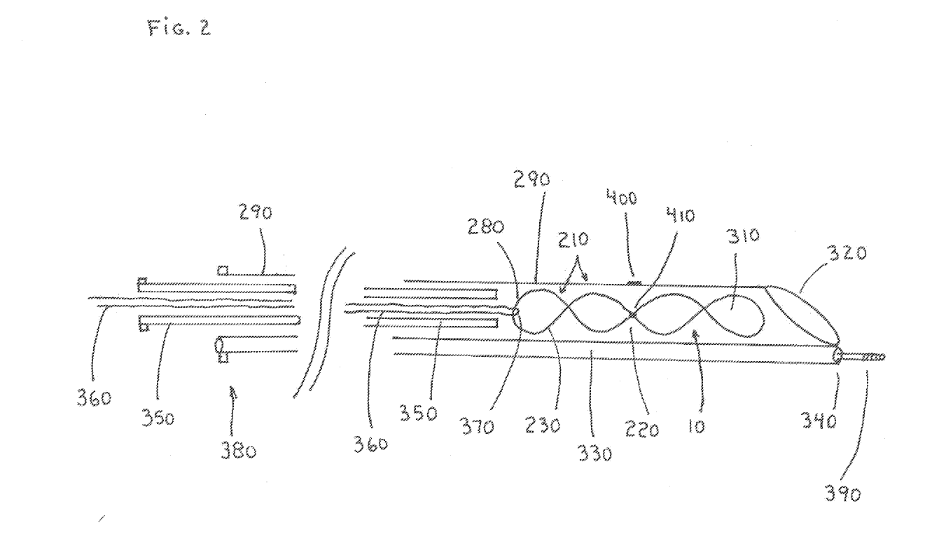

The design of the occlusion device (10) of the present invention allows it to be collapsed into a very low profile and loaded into a delivery sheath (290) as shown in FIG. 2. A very low profile is necessary for the delivery sheath (290) along with a very large flexibility to be able to follow a guidewire (390) from the truncal vessel (20) into the branch vessel (40) which often can be at approximately a 90 degree angle with respect to each other. The frame (300) forms a very low profile in a collapsed state consisting in cross-section essentially of two wires or two frame elements (60) plus the very thin material for the cover (310) as shown in FIG. 2. The frame (300) springs out to form a deployed configuration as shown in FIG. 1A upon release from the deployment sheath of FIG. 2.

The delivery sheath (290) has a beveled distal end (320). A guidewire lumen (330) extends through a separate lumen in the delivery sheath (290) and exits at the distal tip (340) of the bevel. Since the guidewire lumen (330) will tend to require bending of the delivery sheath (290) along a radius of curvature with the guidewire lumen (330) at its center, the occlusion device (10) can be oriented within the delivery sheath (290) such that the inside of the elastically stored perpendicular bend in the frame (300) between the branch portion (50) and the truncal portion (210) at the branch/truncal junction (220) is directed toward the guidewire lumen (330). A pusher tube (350) is located within the delivery sheath (290) in contact with the truncal portion (210) of the occlusion device (10). A control fiber (360) forms a fiber wrap (370) around the truncal frame end and extends through the pusher tube (350) to the proximal end of the delivery sheath (380). The pusher tube (350) is used to apply a force against the occlusion device (10) as it is deployed out of the beveled distal end (320) of the delivery sheath (290) as the delivery sheath (290) is retracted. The control fiber (360) can pull the occlusion device (10) back into the delivery sheath (290) to reposition it if desired. The control fiber (360) is removed by releasing one end and pulling the control fiber (360) out of the fiber wrap (370) around the truncal frame (230). The diameter of delivery sheath (290) for perforator applications may range from 1.5-7 French and could most effectively function in a smaller venous perforator if the delivery sheath (290) profile were 2-4 French. The materials for construction of the delivery sheath (290) include polyethylene, Pebax, Nylon, and other flexible polymeric materials used in the medical device industry.

During use, a guidewire (390), such as approximately a 0.014 inch diameter guidewire (range 0.010-0.035 inch) is first passed using standard percutaneous techniques into the saphenous vein or truncal vessel (20) or deep vein (30) from a location either distally near the ankle or proximally above the lesion site to the location of the incompetent perforator vein to be occluded. The guidewire (390) is advanced under Duplex ultrasound guidance into the incompetent perforator vein or branch vessel (40) and across the incompetent perforator valve and into the deep venous system or other vessel located beyond the perforator vein or branch vessel (40). The delivery sheath (290) containing the nondeployed small diameter occlusion device (10) is passed over the guidewire (390) through the truncal vessel (20) and into the perforator vein under Duplex guidance. A delivery sheath ultrasound marker (400) located on the delivery sheath (290) and on the occlusion device (10) is positioned such that the marker is located near the junction of the truncal vessel (20) with the branch vessel (40).

The ultrasound marker (400) can be a material with significantly different sound reflective or absorptive characteristics than the remainder of the catheter shaft. This would include a gas containing material such as a foam, an irregularly shaped geometry to reflect sound waves in a focused manner back to the Duplex transducer head (like a parabola or other focusing shape), reflecting sound waves in an unfocused manner using an irregular planar geometry such as used with the stealth bomber, a sound generating material such as a piezoelectric source, a material that vibrates from the input of sound waves from the Duplex probe such as an elastic spring with a natural frequency that matches the frequency or a harmonic or multiple of the frequency of the ultrasound transducer, or other ultrasound marker.

The delivery sheath (290) is retracted while the pusher tube (350) is maintained in a stationary position to release the branch portion (50) of the occlusion device (10). Using Duplex guidance the position of an occlusion device ultrasound marker (410) located at the branch/truncal junction (220) located between the branch portion (50) and the truncal portion (210) is assessed to ensure that the device is located at the junction of the branch and truncal vessels or at its appropriate location in the blood vessel; this marker can have similar structure to the delivery sheath ultrasound marker (400). The occlusion device (10) is repositioned if necessary using the delivery catheter and the control fiber (360) to ensure the occlusion device (10) is properly positioned. The truncal portion (210) is then delivered to the truncal vessel (20) by holding the pusher tube (350) stationary and withdrawing the delivery sheath (290) to release the occlusion device (10) into the branch (40) and truncal (20) vessels. The occlusion device (10) expands out to its larger deployed diameter via the elastic energy stored in the branch frame (60) and truncal frame (230) to make contact with the tubular vessel wall (100). The control fiber (360) can be used to pull the occlusion device (10) back into the delivery sheath (290) such that the occlusion device (10) can be repositioned if necessary. Following delivery of the occlusion device (10) to its appropriate location the control fiber (360) can be removed thereby allowing the occlusion device (10) to be fully released into the blood vessel.

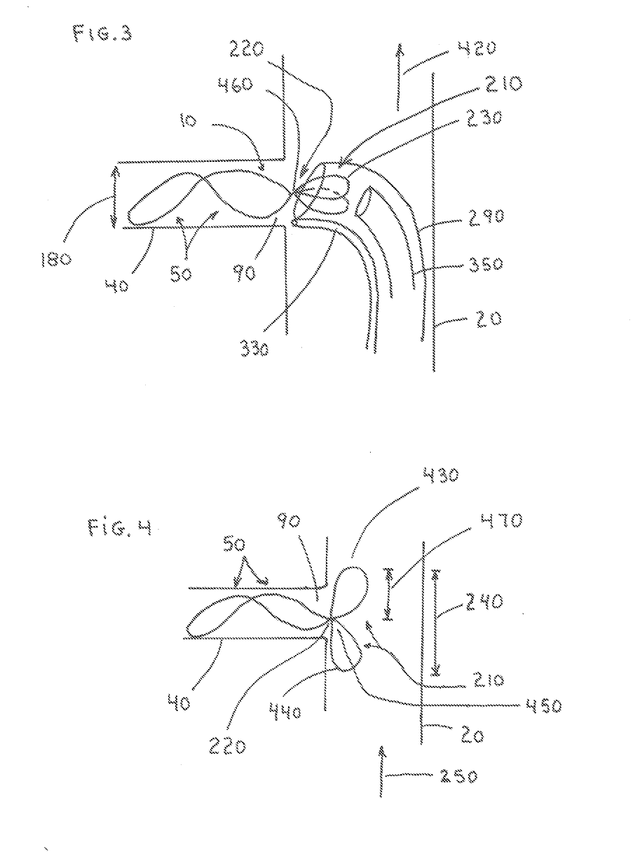

An alternate embodiment for the truncal frame (230) is shown in FIG. 3. The branch portion (50) has been delivered to the perforator vein and the truncal portion (210) is still seen loaded within the delivery sheath (290). The truncal portion (210) has a different configuration than the one shown in FIG. 1A; it is set to be delivered on both the proximal side and distal side (420) of the branch vessel (40). While holding the pusher tube (350) in place, the delivery sheath (290) is withdrawn proximally to allow the truncal portion (210) to be deployed into the truncal vessel (20) as shown in either FIG. 4 or 5. The truncal portion (210) shown in FIG. 4 is comprised of two loops, a truncal distal loop (430) and a truncal proximal loop (440). The branch frame proximal end (90) is attached to the truncal crossover (450) forming a branch/truncal junction (220). This attachment can be swivel type of attachment similar to a shaft that is free to rotate in a bushing. Alternately the connector (460) can be a welded connection or the connection can be contiguous such as a continuous filament of wire or made by laser machining a metal shape similar to forming a stent. The truncal length (240) along the truncal portion axis (250) is longer than the branch portion diameter in a deployed configuration and will thereby prohibit the entry of the truncal portion (210) into the branch vessel (40), eliminating the chances for embolic travel of the branch portion (50) into the deep venous system. The truncal proximal loop (440) and truncal distal loop (430) of the truncal portion (210) are each of a short loop length (470) in the direction of the truncal portion axis (250) to allow them to be deployed into the truncal vessel (20), one distal, and one proximal to the branch vessel (40).

The embodiment of FIG. 5 shows a similar structure for the truncal portion (210) as shown in FIG. 4 except that the truncal frame (230) for the truncal proximal loop (440) and the truncal distal loop (430) is large enough to reach to the opposite truncal vessel wall (480) located opposite to the side with the branch vessel (40). A connector (460) can join the branch portion (50) with the truncal portion (210) or the two portions can be contiguous with each other.

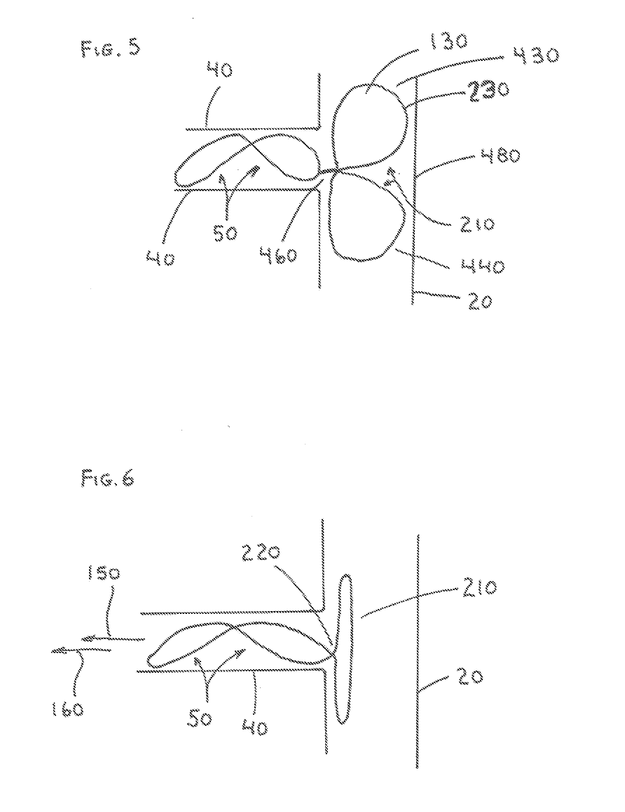

FIG. 6 is yet another embodiment where the truncal portion (210) has a more linear truncal frame (230) to form a more linear element or an elongated loop. The truncal portion (210) with this shape can prevent embolization of the occlusion device (10) into the deep venous system without significant effect on the blood flow in the truncal vein. The connector or branch/truncal junction (220) can be similar to that described for FIGS. 3-5. The truncal portion (210) can be held in a fixed perpendicular configuration to the branch vessel axis (150) or it can be allowed to swivel in a circular manner while maintaining a perpendicular configuration with respect to the branch portion axis (160).

FIGS. 7A and 7B show embodiments where the branch portion length (490) has been shortened to include only one loop and does not contain a figure-eight configuration as described earlier. The single loop is held in a beveled configuration such that the branch frame (60) and branch loop (500) that it forms contact with the branch vessel wall (100) along its perimeter. The single branch loop has a branch frame (60) with a branch cover (200) attached to provide occlusion to the branch vessel (40). The branch portion (50) is connected to a truncal portion (210) that forms a single loop in the truncal vessel (20). The truncal portion (210) has a truncal frame (230) that makes contact with the opposite truncal vessel wall (480) opposite to the branch vessel (40) to ensure that the branch portion (50) is not able to move or embolize into the truncal vessel (20). The truncal length (240) is larger than the diameter of the branch vessel to prevent embolization of the occlusion device (10) into the branch vessel (40).

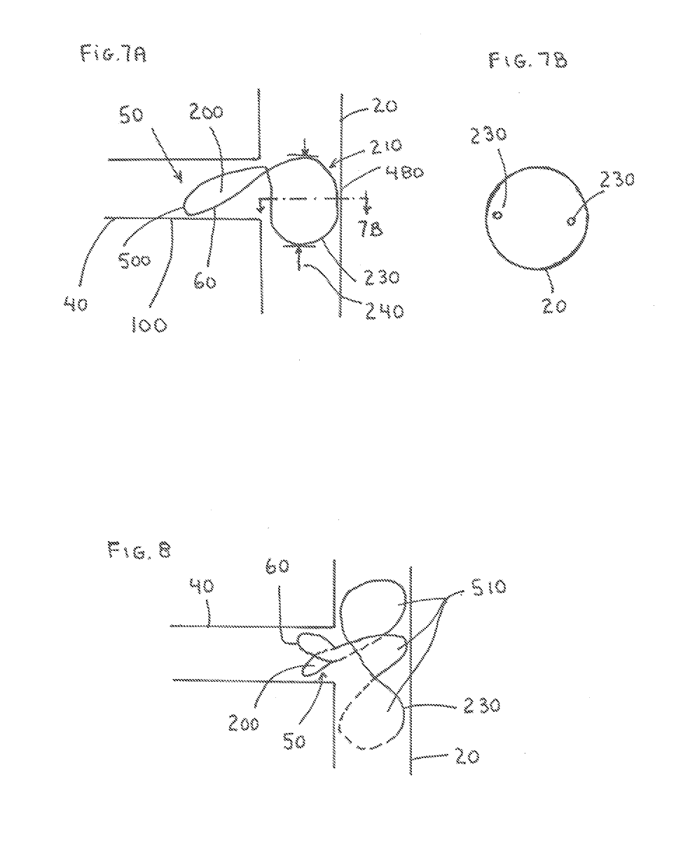

FIG. 8 shows one more embodiment where the branch portion (50) is even shorter than that shown in the embodiment of FIGS. 7A and 7B. Here the branch frame (60) of the branch portion (50) of FIGS. 7A and 7B has been essentially folded such that it does not extend appreciably into the perforator vein or branch vessel (40). A branch cover (200) is attached to the branch frame (60). The branch frame (60) extends contiguously into a truncal frame (230) that forms three truncal loops (510). The truncal frame (230) does not have a truncal cover (270) so that blood flow can occur freely in the truncal vein.

It is understood that each of the embodiments described in FIGS. 1-8 are not limited to the configurations shown in these drawings; the branch portion (50) can have a figure-eight configuration with two loops, or one loop, or a folded loop as shown, but it can also have three or more loops or somewhat modified configuration as long as the branch frame (60) makes contact with the branch vessel (40) along its entire perimeter (170) and can adapt to changes in the vessel diameter and has an occluding branch cover (200). Similarly, the truncal portion (210) can have any of the frame shapes shown along with any of the branch shapes shown or described or modifications of these frame designs. The truncal portion (210) is required to have a dimension along the truncal axis, approximately perpendicular to the axis of the branch portion axis (160), that is larger than the branch portion effective diameter (180) after it has expanded into contact with the branch vessel (40). An ultrasound marker (410) can be located on the occlusion device (10) preferably near the branch/truncal junction (220).

It is further understood that the embodiments described in FIGS. 1-8 also can be applied to occlude truncal vessels without consideration for treating a branch vessel (40). The direction of the truncal portion axis (250) being perpendicular to the branch portion (50) along with the truncal portion length being greater than the branch portion effective diameter (180) and the branch vessel (40) diameter will reduce the likelihood for embolization of the occlusion device (10) in generally straight or tortuous truncal vessel.

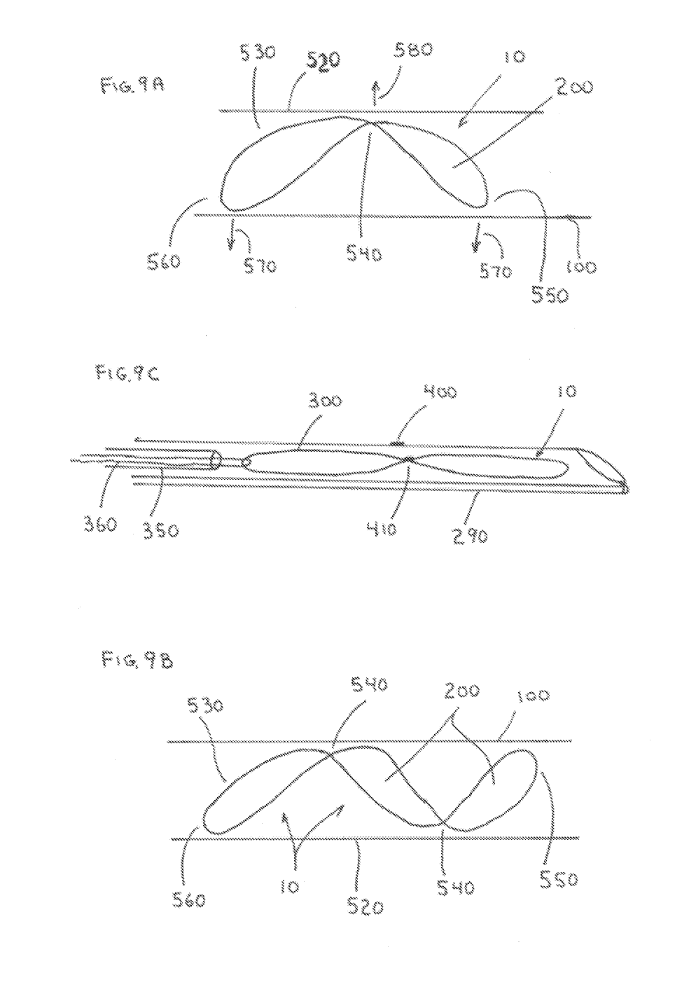

FIGS. 9A-9C show additional embodiments for an occlusion device (10) that has one portion, similar to the branch portion (50) described in the embodiments of FIGS. 1-8. The embodiments of FIGS. 9A and 9B are intended to be placed in a long length perforator or branch vessel (40) or directly into a truncal vessel (20) or tubular vessel (520) that is incompetent. For an incompetent truncal or long branch vessel (40), the occlusion device (10) can have two loops (530) such as a figure-eight configuration as shown in FIG. 9A or it can have three loops (530) as shown in FIG. 9B, or it can have more than three loops or less than two (not shown). The figure-eight frame of FIG. 9A or the three loop frame configuration of FIG. 9B are not planar but instead are curved to place the proximal and distal ends into contact with the vessel wall (100) and also place the crossovers (540) into contact with the vessel wall (100). The frame distal (550) and proximal (560) ends of the occlusion device (10) will push outwards with a force (570) against the vessel wall (100). Also, the crossovers (540) will push outward against the vessel wall (100) with an opposing force (580). These outward forces are able to force the perimeter (170) of the frame against the vessel wall (100) to form a good seal (190) and also to prevent the occlusion device (10) from migrating in the vessel. The occlusion device (10) will adjust to a changing diameter for the vein by altering the bevel angle (125) for the loops as discussed earlier in FIG. 1A. The elastic energy stored in the frame causes the frame to form open loops that are not planar and are pushed into contact with the vessel wall (100) to create a seal (190) of the occlusion device (10) with the vessel wall (100). The occlusion device (10) would have a branch cover (200) to occlude flow and an ultrasound marker (410) to provide positioning capability. The construction materials have been described earlier in FIGS. 1-8.

The occlusion device (10) of FIGS. 9A and 9B can be delivered in a manner similar to that described for the previous embodiments. The occlusion device (10) for truncal or perforator occlusion can be collapsed and fit within a delivery sheath (290) that is approximately 1.5-9 French in diameter or larger (preferably 2-7 French diameter) and travels over a 0.014 guidewire (390) or larger guidewire for truncal occlusion applications. Truncal vessel diameters can be larger than the diameters of branch vessels ranging from 3 mm to approximately 20 mm in diameter. Entry into a truncal superficial vessel can be at the ankle with antegrade passage or more proximally into a truncal vessel (20) with retrograde passage. A pusher tube (350) is used to hold the occlusion device (10) while the delivery sheath (290) such as that described in FIG. 2 is withdrawn. A control fiber (360) is used to pull the occlusion device (10) back into the delivery sheath (290) to reposition it if necessary.

It is understood that while most of the description used in this application has been directed to venous applications, the occlusion device (10) can equally be used in arteries and tubular vessels of sizes that differ from those of the venous system. In this case the dimensions of the branch and truncal portions (210) may change without deviating from the present invention.

The delivery sheath (290) shown in FIG. 9C can be used to deliver any of the device embodiments shown in the present application into the perforator vein from either the deep vein (30), from a superficial vein, or from a direct access through the skin and into the perforator vein. For providing direct access to the perforator vein, transcutaneously, or directly through the skin, a needle can be advanced into the lumen of the perforator under Duplex ultrasound guidance. The access site for entry through the skin can be directly above the perforator vein or at a location on the skin that is approximately within three to six inches of access into the lumen of the perforator vein. A guidewire (390) can then be advanced through the needle and into the perforator vein. The delivery sheath (290) containing the occlusion device (10) can then be advanced over the guidewire (390) and into the lumen of the perforator vein where it can then be deployed using similar techniques as described herein. The occlusion device (10) can be deployed into the perforator vein, the truncal vein, a tributary vein, an accessory vein or a combination of such veins.

Reference numerals that are common to more than one embodiment of the present invention are intended to describe elements of the invention having a similar structure and with a similar description as that already described. The various aspects of different embodiments are understood to be equally applied to all embodiments of the invention.

1. An occlusion device for blocking flow in a tubular blood vessel of the body, the occlusion device comprising;
A. a first portion having a first frame formed from an elastic material configured into at least one first portion loop formed from a single frame element, said first portion loop being held at a bevel with respect to the axial direction of the blood vessel to form a beveled angle, said first portion loop forming a contact with the tubular blood vessel along a perimeter of said first portion loop, said first portion loop having an effective diameter in a deployed configuration that is substantially equal to the diameter of the blood vessel,
B. a first portion cover attached to said first frame along said perimeter of said first portion loop and extending over said effective diameter of said first portion loop, said first portion cover formed from a material that substantially prevents blood flow through said first cover, said first frame forming a seal with the blood vessel to prevent blood flow through the blood vessel,
C. said first portion being delivered to the tubular vessel of the body in a small diameter configuration and expandable out to said effective diameter in its deployed configuration.

2. The occlusion device of claim 1 wherein said first frame is comprised entirely of a single continuous frame member that is formed into said first portion loop.

3. The occlusion device of claim 1 wherein said first portion further comprises;
A. a first frame that forms two first portion loops, said two first portion loops forming a first figure-eight configuration, said first figure-eight configuration being nonplanar and having a crossover in a deployed configuration, said crossover making contact with the wall of the tubular blood vessel,
B. each of said two first portion loops forming a beveled angle with respect to the axial direction of the tubular blood vessel,
C. each of said two first portion loops forming a contact with the tubular blood vessel along a perimeter of each of said two first portion loops forming a seal between said first portion loops and the blood vessel, said first portion loops having an effective diameter that is substantially equal to the diameter of the tubular blood vessel,
D. each of said two first portion loops being comprised of said first cover, said first cover being attached to said first frame along a perimeter of each of said two first portion loops and extending over the effective diameter of each of said two first portion loops, said first portion cover formed from a material that substantially prevents blood flow through said first cover, each of said two first portion loops thereby preventing blood flow through said blood vessel.

4. The occlusion device of claim 3 wherein said two first portion loops are formed from a single contiguous frame element.

5. The occlusion device of claim 1 further comprising a second portion, said second portion comprising;
A. a second frame formed from an elastic material attached to said first frame, said second frame having a length in a direction perpendicular to an axial direction of said first portion that is greater than said effective diameter of said first portion.

6. The occlusion device of claim 5 wherein said second frame has a second figure-eight configuration, said second figure-eight configuration of said second frame being nonplanar in a deployed configuration.

7. The occlusion device of claim 3 wherein said second frame has a second portion cover, said second portion cover formed from a material that is attached to said second frame, said second portion cover being formed from a material that substantially prevents blood flow from passing through it, said second portion cover making contact with the vessel wall along its perimeter to form a seal with the vessel wall, wherein blood flow is unable to pass through said second frame and through the blood vessel.

8. The occlusion device of claim 5 wherein said first frame and said second frame are formed from a single contiguous frame element.

9. The occlusion device of claim 5 wherein said first frame and said second frame are joined together by a connector.

10. The occlusion device of claim 5 wherein said occlusion device in a nondeployed configuration has a cross-section for the frame that consists solely of two frame elements, wherein the profile of said occlusion device is formed substantially by the cross-section of said two frame elements.

11. The occlusion device of claim 10 wherein said frame element is an elastic wire formed from an elastic metal.

12. The occlusion device of claim 5 further comprising a delivery system, said delivery system comprising;
A. a delivery sheath for holding said occlusion device in a small diameter non-deployed configuration for delivery to the tubular blood vessel,
B. a pusher tube to push against said occlusion device during deployment of said occlusion device into the tubular blood vessel from said smaller diameter configuration to said larger deployed configuration,
C. a control fiber looped around said first or second frame to pull said occlusion device back into said delivery sheath after it has been at least partially deployed to said larger effective diameter.

13. The occlusion device of claim 1 further comprising an ultrasonic marker located on said first frame, said ultrasound marker taken from a group comprising an ultrasound reflecting material and an ultrasound generating material.

14. The occlusion device of claim 12 further comprising an ultrasonic marker located on said delivery sheath, said ultrasound marker taken from a group comprising an ultrasound reflecting material and an ultrasound generating material.

15. An occlusion device for delivery to a tubular vessel of the body in a smaller diameter configuration and expandable to a larger diameter, said occlusion device comprising,
A. a first loop portion and a second loop portion,
B. said first loop portion being formed from a first frame and said second loop portion being formed from a second frame, at least one of said first and second frames making contact with the tubular vessel along their perimeters,
C. said first loop portion having an axis that extends with a substantial angle with respect to an axis of said second loop portion,
D. at least one of said loop portions having a cover, said loop portion having a cover forming a seal with the tubular vessel along said perimeter, said cover preventing flow of fluid through said tubular vessel across said occlusion device, said seal preventing flow through the tubular vessel.

16. The occlusion device of claim 15 wherein said first frame has a cross-sectional area in its nondeployed configuration that is comprised solely of two frame elements.

17. The method for deploying an occlusion device for blocking flow in a tubular vessel of the body, the occlusion device comprising a first portion having a first frame formed from an elastic material configured into at least one first portion loop, and a first cover attached to said first frame along a perimeter of said first portion loop to form a seal with the tubular vessel, said first cover formed from a material that substantially prevents blood flow through said first cover, said first portion being delivered to the tubular vessel of the body in a small diameter configuration and expandable out to an effective diameter in its deployed configuration to form a seal between said perimeter and the tubular vessel to prevent blood flow through the tubular vessel.

18. The method of claim 17 wherein said occlusion device is delivered from a delivery catheter that is advanced directly into the lumen of a branch vessel adjoining the tubular vessel from a puncture site on the skin that is approximately adjacent or above the branch vessel.
